(19) United States
(12) Patent Application Publication
(10) Pub. No.: US 2025/0001192 A1
(43) Pub. Date: Jan. 2, 2025

(54) TRANSDUCERS FOR DELIVERY OF TUMOR TREATING FIELDS AND CAPABLE OF REDUCING CREASING

(71) Applicant: Novocure GmbH, Root D4 (CH)

(72) Inventors: Noa HALAVEE, Tel Aviv (IL); Elie YAACOBI, Haifa (IL)

(73) Assignee: Novocure GmbH, Root D4 (CH)

(21) Appl. No.: 18/757,028

(22) Filed: Jun. 27, 2024

Related U.S. Application Data
(60) Provisional application No. 63/524,586, filed on Jun. 30, 2023.

(51) Int. Cl. A61N 1/40; A61N 1/04

(52) U.S. Cl. CPC A61N 1/40; A61N 1/0408; A61N 1/0476

(57) ABSTRACT

A transducer apparatus for delivering tumor treating fields to a subject's body, the transducer apparatus comprising: a substrate; an array of at least one electrode disposed on the substrate, the array configured to be positioned over the subject's body with a face of the array facing the subject's body, the array capable of delivering tumor treating fields to a subject's body; and a layer of anisotropic material positioned on a skin-facing side of the array; wherein, when viewed from a direction perpendicular to the face of the array, the layer of anisotropic material comprises an interior slit extending substantially along a longitudinal direction of the layer of anisotropic material, the interior slit is surrounded by anisotropic material, and no electrodes are in the interior slit.

### CROSS-REFERENCE TO RELATED APPLICATIONS

This application claims priority to U.S. Provisional Application No. 63/524,586, filed Jun. 30, 2023, which is incorporated herein by reference in its entirety. This application is related to U.S. Provisional Application No. 63/524,561, filed Jun. 30, 2023, which is incorporated herein by reference in its entirety.

### BACKGROUND

Tumor treating fields (TTFields) are low intensity alternating electric fields within the intermediate frequency range (for example, 50 kHz to 1 MHZ), which may be used to treat tumors as described in U.S. Pat. No. 7,565,205. In current commercial systems, TTFields are induced non-invasively into the region of interest by electrode assemblies (e.g., arrays of capacitively coupled electrodes, also called electrode arrays, transducer arrays or simply “transducers”) placed on the patient's body and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIGS. 1A, 1B, 2A, and 2B depict examples of transducers located on a subject's body for delivery of TTFields.

FIG. 3 depicts a top view of two example transducers.

FIG. 4A depicts a top view of two example transducers.

FIG. 4B depicts an example transducer of FIG. 4A applied to an example breast site.

FIG. 5 depicts a top view of two example transducers.

FIG. 6 depicts a top view of an example transducer.

FIG. 7 depicts a top view of an example transducer.

FIG. 8 depicts two transducers after removal from a subject.

FIG. 9 depicts two transducers after removal from a subject.

### DESCRIPTION OF EMBODIMENTS

This application describes exemplary transducers (or transducer apparatuses) used to deliver TTFields to a subject's body for treating one or more cancers.

The inventors have recognized that a transducer used to deliver TTFields to a subject may exhibit creasing when applied to and worn by a subject. The creasing of the transducer may occur and even be more pronounced when the transducer is located on a curved part of the subject (e.g., a breast or a head) and/or on a part of a subject having substantial movement (e.g., an upper torso). Although not limited to transducers comprising a layer of anisotropic material (such as, for example, a sheet of graphite), the issue of creasing of the transducer may be more pronounced in this case because of the relative inflexibility of the layer of anisotropic material. The creasing of the transducer may affect the ability of the transducer to deliver a recommended dosage of TTFields to the subject.

The inventors have recognized that a need exists to reduce creasing of a transducer used to deliver TTFields to a subject. The inventors have discovered that a transducer with one or more interior slits may reduce creasing of the transducer.

Different types of transducers are described herein. Each of the embodiments disclosed herein may be used for one or more of the transducer types described herein.

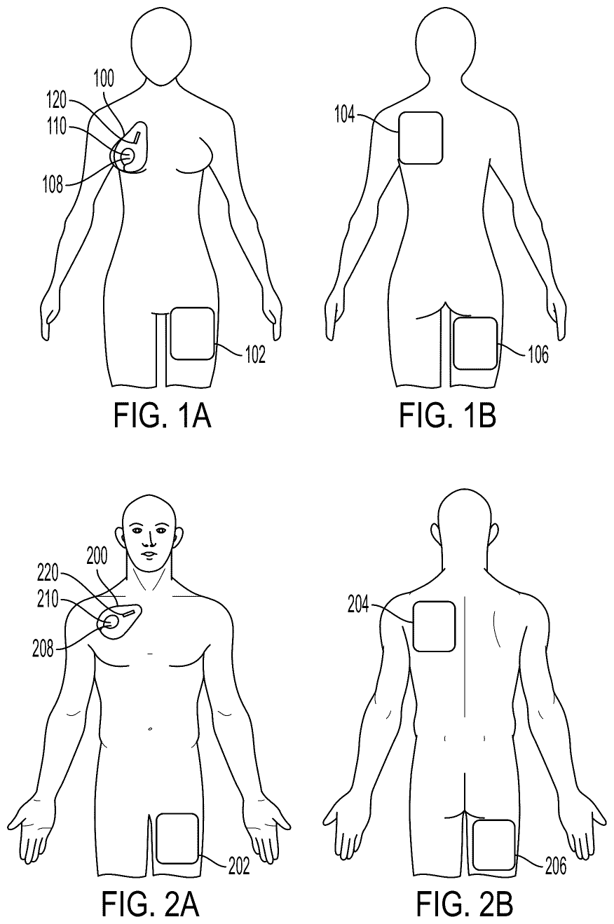

FIGS. 1A and 1B depict an example of transducers positioned at locations on a subject's body for delivery of TTFields. FIG. 1A depicts a first transducer 100 located on the front of the subject's right breast and a second transducer 102 located on the front of the subject's left thigh. FIG. 1B depicts a third transducer 104 located on the left side of the subject's upper back and a fourth transducer 106 located on the back of the subject's right thigh. Each of the transducers 100, 102, 104, and 106 may include one or more electrode elements located on a surface that is flexible for contouring the transducer to the subject's body. The transducers 100, 102, 104, and 106 may be capable of delivering TTFields to the subject's body.

Similarly, FIGS. 2A and 2B depict another example of transducers positioned at locations on a subject's body for delivery of TTFields. FIG. 2A depicts a first transducer 200 located on the front of the subject's right thorax and a second transducer 202 located on the front of the subject's left thigh. FIG. 2B depicts a third transducer 204 located on the left side of the subject's upper back and a fourth transducer 206 located on the back of the subject's right thigh. Each of the transducers 200, 202, 204, and 206 may include one or more electrode elements located on a surface that is flexible for contouring the transducer to the subject's body. The transducers 200, 202, 204, and 206 may be capable of delivering TTFields to the subject's body.

Transducers arranged on a subject's torso (as shown in FIGS. 1A-2B) are capable of applying TTFields to a tumor in the subject's thorax or abdomen. The transducers may be located at various other combinations of locations on the subject's torso than those of FIGS. 1A-2B.

FIGS. 1A and 1B and FIGS. 2A and 2B illustrate an assembly for applying TTFields to a subject's body while avoiding at least one area with an anatomic feature or device. For example, in FIG. 1A, the surface of the transducer 100 is shaped and adapted for contouring over a breast of the subject's body while avoiding a nipple 108 of the subject's body. In some embodiments, once placed on the subject's body, a substantially circular opening 110 coincides with the nipple 108 of the subject's body, such that no electrodes of the transducer are located over the nipple 108. The transducer 100 may include an interior slit 120, which may help to reduce creasing of the transducer 100 when affixed to the subject.

As another example, in FIG. 2A, the surface of the transducer 200 is shaped and adapted for contouring to avoid a chemotherapy port 208 on the subject's body. In particular, the surface of the transducer 200 is adapted to be positioned on the subject's body such that a substantially circular opening 210 of the transducer 200 coincides with a location on the subject's body having the chemotherapy port 208. The transducer 200 may include an interior slit 220, which may help to reduce creasing of the transducer 200 when affixed to the subject. In another example, the surface of the transducer 200 may be positioned on the subject's body with two opposing portions of the transducer surface spaced apart to straddle a location on the subject's body having the chemotherapy port 208. No electrodes of the transducer 200 may be located over the chemotherapy port 208. Chemotherapy ports 208 are often inserted into a subject's body prior to the subject receiving TTFields treatment. The transducers disclosed herein may enable the application of TTFields to a region of interest in the subject's thorax or abdomen without interfering with or being affected by the subject's chemotherapy port 208.

Turning back to FIGS. 1A and 1B, one or more other transducers 102, 104, and 106 may have a different shape than the transducer 100. As illustrated, for example, each of the second, third, and fourth transducers 102, 104, and 106 of the assembly has a different shape than the first transducer 100. In some embodiments, each of the second, third, and fourth transducers 102, 104, and 106 may have the same or a substantially similar shape to each other. As illustrated, the surface of at least one of the transducers 102, 104, and 106 may have a substantially convex shape. More particularly, the surface of at least one of the transducers 102, 104, and 106 may have a rectangular, substantially rectangular with rounded corners (as illustrated), circular, oval, ovaloid, ovoid, or elliptical shape. Similar situations may apply to the transducers 200, 202, 204, and 206. In particular, the transducer 200 illustrated in FIG. 2A may have similar shape as the transducer 100 illustrated in FIG. 1A, and the transducers 202, 204, and 206 illustrated in FIGS. 2A and 2B may have similar shapes as the transducers 102, 104, and 106 shown in FIGS. 1A and 1B.

In other embodiments, one or more of the other transducers 102, 104, and 106 may have a surface having the same shape or a mirror image shape of the transducer 100 of FIGS. 1A and 1B. Similarly, one or more of the other transducers 202, 204, and 206 may have a surface having the same shape or a mirror image shape compared to the transducer 200 of FIGS. 2A and 2B.

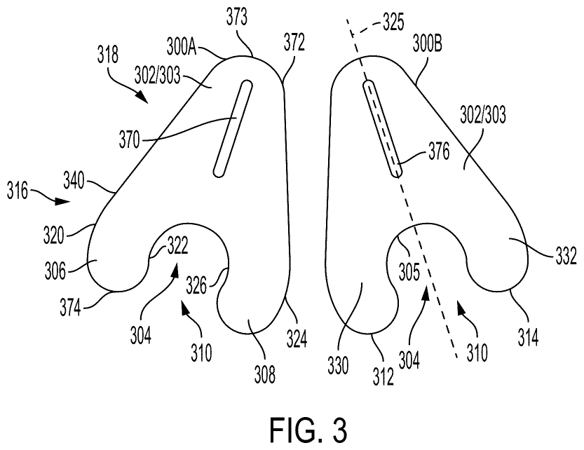

FIG. 3 depicts a top view of two example transducers 300A and 300B. The transducers 300A and 300B have the same shape and features. The transducers 300A and 300B are shown in different orientations, as may be applied to the breasts of a subject. The transducers 300A, 300B may each include a substrate 302, having a first side to face the subject and a second side opposite the first side. One or more electrodes (not shown) may be attached to the first side of the substrate 302, optionally with an adhesive layer between the first side of the substrate 302 and the one or more electrodes.

The transducer 300A, 300B (and any other transducers disclosed or discussed herein) may include any of the features discussed herein and may include any desired number of electrode elements (e.g., one or more electrode elements). For example, the transducer may include one, two, three, four, five, six, seven, eight, nine, ten, or more electrode elements (e.g., twenty electrode elements). Various shapes, sizes, and materials may be used for the electrode elements. For example, the electrode elements may be triangular, square, rectangular, circular, oval, ovaloid, ovoid, or elliptical in shape or substantially triangular, substantially square, substantially rectangular, substantially circular, substantially oval, substantially ovaloid, substantially ovoid, or substantially elliptical in shape. Any constructions for implementing the transducer (or electric field generating device) for use with embodiments of the invention may be used as long as they are capable of delivering TTFields to the subject's body. The transducer may be conductive or non-conductive. In some embodiments, an AC signal may be capacitively coupled into the subject's body. In certain embodiments, at least one electrode (or electrode element) of the transducer(s) (e.g., transducer 300A, 300B) adapted to generate an alternating electric field can include a ceramic disk as a dielectric layer. The one or more electrodes (or electrode elements) of the transducer 300A, 300B may be non-ceramic dielectric materials positioned over a plurality of flat conductors. Examples of non-ceramic dielectric materials positioned over flat conductors may include polymer films disposed over pads on a printed circuit board or on substantially planar pieces of metal. In some embodiments, such polymer films have a high dielectric constant, such as, for example, a dielectric constant greater than 10.

The transducer 300A, 300B may be configured to be positioned over a subject's body with a face of an array of at least one electrode facing the subject's body. In some embodiments, the transducer 300A, 300B may be substantially planar. In some embodiments, the transducer 300A, 300B may be substantially planar prior to being located, applied, or affixed to a subject.

In some embodiments, the transducer 300A, 300B (and any other transducers disclosed or discussed herein) may include a layer of anisotropic material, as discussed further herein below. For example, the layer of anisotropic material may overlay the at least one electrode on the skin-facing side of the array of the at least one electrode (such that the layer of anisotropic material may be facing the first side of the substrate 302). In some embodiments, the layer of anisotropic material may be present as, or may compromise, a laminate having a layer of conductive adhesive, a layer of anisotropic material, and a layer of conductive adhesive. In some embodiments, the anisotropic material may be a sheet of graphite. In some embodiments, the layer of anisotropic material may be a sheet of pyrolytic graphite, graphitized polymer film, or graphite foil made from compressed high purity exfoliated mineral graphite. In some embodiments, the layer of anisotropic material may take the same or similar shape as that of the substrate 302, and may be of a similar size or slightly smaller than that of the substrate. Moreover, in some embodiments, a layer of adhesive may be present between the substrate and the layer of anisotropic material. For simplicity, FIGS. 1-5 and 7 do not show both the substrate and the layer of anisotropic material, although both may be present in one or more (or all) of these embodiments. For embodiments without a layer of anisotropic material, the shape of the main body of the transducer may (or may not) reflect the shape of the substrate (e.g., 302, 402, 502, 702 in FIGS. 3-5 and 7). In these Figures (FIGS. 3-5 and 7), it is to be understood that although the slits are shown as slits in the substrate layer, these embodiments apply equally to slits in a layer of anisotropic material of a similar (or same) shape, or to slits passing through both the substrate and the layer of anisotropic material. FIGS. 6, 8 and 9 show the layer of anisotropic material as 603, 803 and 903, respectively, the outer perimeter of which may (as in FIGS. 6, 8 and 9), or may not, contour the shape of the main body of the transducer. Similarly, in describing the process of deforming the planar transducer to produce the non-planar transducer, it is to be understood that the substrate and the layer of anisotropic material may have the same or similar shape (overlapping one on the other) and may be adhered to one another. Accordingly, in some embodiments, folding a first portion of the substrate over a second portion of the substrate may include folding both layers (the substrate and the layer of anisotropic material) simultaneously.

The transducer 300A, 300B may include an interior slit 370 situated within an interior of the substrate 302 (and/or within the layer of anisotropic material 303, if present). In some embodiments, the interior slit 370 may be an opening through the substrate 302 (or layer of anisotropic material 303). In some embodiments, the interior slit 370 may be located entirely within a perimeter of the substrate 302 (or layer of anisotropic material 303). In some embodiments, the interior slit 370 may be a perforation through the substrate 302 (or layer of anisotropic material 303). In some embodiments, no electrodes are in the interior slit 370.

In some embodiments, the interior slit 370 may be positioned along a centerline 325 running through a longest dimension of the substrate 302 (or layer of anisotropic material 303). The centerline 325 may divide the substrate 302 (or layer of anisotropic material 303) into a first portion 330 and a second portion 332. In some embodiments, the centerline 325 may divide the substrate 302 (or layer of anisotropic material 303) into a first portion 330 and a second portion 332 of approximately same sizes, approximately same shapes, and having reflectional symmetry. The interior slit 370 may have reflectional symmetry about the centerline 325. In some embodiments, the interior slit 370 may extend from below a top edge 372 (which includes an uppermost point 373) of the substrate 302 (or layer of anisotropic material 303) to above a bottom edge 374 (which includes a concave edge 305 of an opening 304, a first end 312, and a second end 314). In some embodiments, where the interior slit 370 is an opening through the substrate 302, the interior slit 370 may be situated between the uppermost point 373 and the concave edge 305 and may be surrounded by the substrate 302. In some embodiments where the transducer includes a layer of anisotropic material, the interior slit 370 may be an opening through the layer of anisotropic material. In some such cases, the interior slit 370 may be situated between the uppermost point 373 and the concave edge 305 and may be surrounded by the anisotropic material. In some embodiments, the interior slit 370 is an opening through both the substrate and through the anisotropic material. In some such cases, the interior slit 370 may be situated between the uppermost point 373 and the concave edge 305 and may be surrounded both by the substrate and by the anisotropic material.

When viewed from a direction perpendicular to the face of the transducer 300A, 300B (or the face of the array of at least one electrode of the transducer 300A, 300B), the interior slit 370 may be substantially rectangular in shape (e.g., rod-shaped) or rectangular with rounded vertices, but the interior slit 370 may have other shapes compatible with the embodiments disclosed herein. In some embodiments, a centroid 376 of the substrate 302 (or of the layer of anisotropic material 303) may be situated within the interior slit 370. In some embodiments, a centroid of the transducer 300A, 300B may be situated within the interior slit 370. In some embodiments, the substrate 302 (or layer of anisotropic material 303) includes only one interior slit. In some embodiments, the substrate 302 (or layer of anisotropic material 303) does not include an interior slit extending substantially perpendicular to the longitudinal direction of the substrate 302 (or layer of anisotropic material 303) and/or the centerline 325. In some embodiments, the substrate 302 (or layer of anisotropic material 303) comprises a plurality of interior slits 370 extending substantially in parallel and substantially along the longitudinal direction of the substrate (or layer of anisotropic material 303). In some embodiments, the substrate 302 (or layer of anisotropic material 303) includes two discontinuous non-parallel interior slits.

In some embodiments, when viewed from a direction perpendicular to the face of the transducer, the interior slit 370 includes approximately 0.5% to approximately 10% of a surface area of the substrate 302 (or approximately 0.5% to approximately 10% of a surface area of the layer of anisotropic material 303). In further embodiments, the interior slit 370 includes approximately 1% to approximately 5% of a surface area of the substrate 302 (or approximately 1% to approximately 5% of a surface area of the layer of anisotropic material 303). In some embodiments, the interior slit 370 is approximately 0.5 mm to approximately 10.0 mm wide. In further embodiments, the interior slit 370 is approximately 1.0 mm to approximately 5.0 mm wide. In some embodiments, the interior slit 370 is approximately 1.0 cm to approximately 10.0 cm long. In some embodiments, the interior slit 370 is approximately 3.0 cm to approximately 6.0 cm long.

In some embodiments, when viewed from a direction perpendicular to a face of the transducer 300A, 300B, the transducer may have a substantially pear-shaped or rounded triangular-shaped surface. In some embodiments, when viewed from a direction perpendicular to a face of the transducer 300A, 300B, the substrate 302 (or layer of anisotropic material 303) may have a substantially pear-shaped or rounded triangular-shaped surface.

In some embodiments, when viewed from a direction perpendicular to the face of the array and when the transducer 300A, 300B is substantially planar, the substrate 302 (or layer of anisotropic material 303) may have an opening 304 located towards a wider portion 316 than a narrower portion 318 of the substrate 302 (or layer of anisotropic material 303). In some embodiments, no electrodes are in the opening 304. The opening 304 may have at least one concave edge 305 defining the opening 304 between two opposing portions, namely the first portion 330 and the second portion 332, of the substrate 302 (or layer of anisotropic material 303). The concave edge 305 may include a substantially C-shaped concave surface.

In some embodiments, when viewed from a direction perpendicular to the face of the array and when the transducer 300A, 300B is substantially planar, the transducer 300A, 300B may include a first end portion 306 separated from a second end portion 308 by a gap 310. The gap 310 may be located closer to the wider portion 316 than the narrower portion 318 of the substrate 302 (or layer of anisotropic material 303). The centerline 325 may run through a longest dimension of the substrate 302 (or layer of anisotropic material 303) and through a center of the gap 310. The substrate 302 (or layer of anisotropic material 303) may have reflectional symmetry, and the reflectional symmetry of the substrate 302 (or layer of anisotropic material 303) may be about the centerline 325.

The substrate 302 (or layer of anisotropic material 303) may have two opposing ends (or opposing sides), namely the first end 312 and the second end 314. The gap 310 may be defined as being between the two opposing ends 312, 314 of the substrate 302 (or layer of anisotropic material 303). The first end 312 may include a convex edge, and the second end 314 may include a convex edge.

The first end portion 306 may have a first edge 320 and a second edge 322. The first edge 320 may define a portion of an exterior edge 340 of the substrate 302 (or layer of anisotropic material 303) and may be convex shaped. The second edge 322 may define a portion of concave edge 305 of the opening 304 and may be concave shaped. The second end portion 308 may have a first edge 324 and a second edge 326. The first edge 324 may define a portion of the exterior edge 340 of the substrate 302 (or layer of anisotropic material 303) and may be convex shaped. The second edge 326 may define a portion of the concave edge 305 of the opening 304 and may be concave shaped. The opening 304 may have a partially circular, nearly circular, substantially partially circular, or substantially nearly circular edge defined by the first edge 322 and the second edge 326.

In some embodiments, the first portion 330 and the second portion 332 may be used to define the opening 304. In some embodiments, the first end portion 306 and the second end portion 308 may be used to define the opening 304. In some embodiments, the first end portion 306 and the second end portion 308 may be used to define the gap 310. In some embodiments, the first portion 330 and the second portion 332 may be closer to the narrower portion 318, and the first end portion 306 and the second end portion 308 may be closer to the wider portion 316. In some embodiments, the first portion 330 and the first end portion 306 may at least partially overlap, and in some embodiments, the second portion 332 and the second end portion 308 may at least partially overlap.

In some embodiments, the first end portion 306 and the second end portion 308 may each include a portion of the array of at least one electrode. In other embodiments, only one of the first end portion 306 or the second end portion 308 includes a portion of the array of at least one electrode. In some embodiments, the first end portion 306 and the second end portion 308 are part of a single continuous substrate 302 (or layer of anisotropic material 303). In other embodiments, the first end portion 306 and the second end portion 308 are located on two separate discontinuous sections of substrate 302 (or layer of anisotropic material 303).

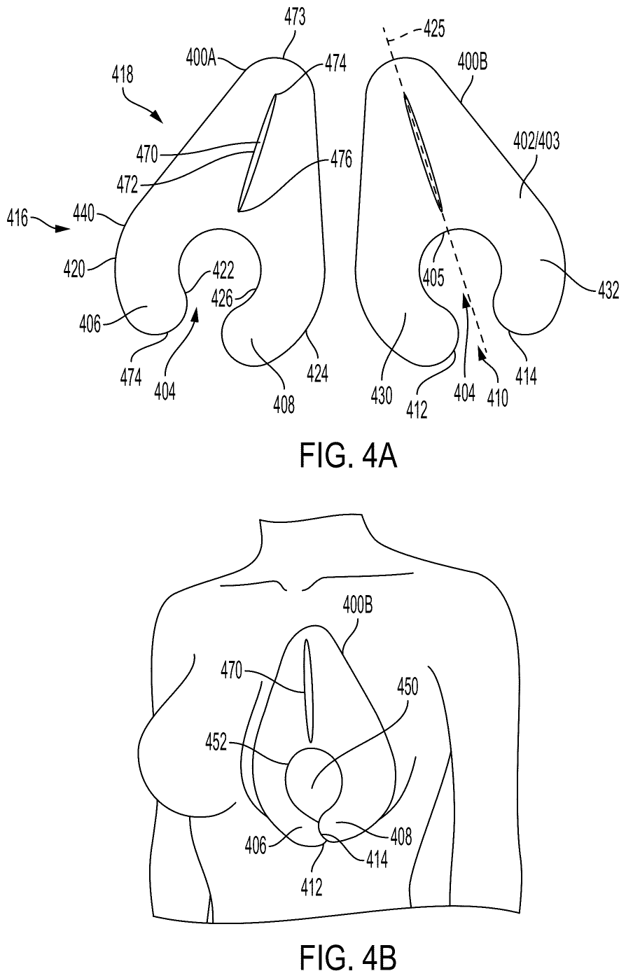

The transducer 300A, 300B may be capable of being deformed from being substantially planar to being substantially non-planar, such as being deformed to be shaped as a substantially truncated elliptical paraboloid or substantially truncated oblique cone or the like. As a non-planar shape, the transducer 300A, 300B may be substantially conical, such as substantially truncated elliptical paraboloid or substantially truncated oblique cone. In being deformed, the first end 312 and the second end 314 are brought together to form a three-dimensional shape, such as a substantially truncated elliptical paraboloid or a substantially truncated oblique cone (see, for example, FIG. 4B). The three-dimensional shape may have a substantially circular opening formed in part by the opening 304 (as viewed in the planar transducer, FIG. 3). When placed on a subject, a nipple or a chemotherapy port may reside within the substantially circular opening of the three-dimensional shape.

FIG. 4A depicts a top view of two example transducers 400A and 400B. The transducers 400A and 400B have the same shape and features. The transducers 400A, 400B are similar to the transducers 300A, 300B, and the labelling scheme for transducers 400A, 400B follows the above labelling scheme for transducers 300A, 300B. As such, the features labelled 4xx for transducers 400A, 400B are similar to the features labelled 3xx for transducers 300A, 300B. The transducers 400A, 400B are similar to the transducers 300A, 300B but differ by having longer first and second end portions 406, 408 compared to first and second end portions 306, 308 and by having an interior slit 470 of different shape. The interior slit 470 may be wider in a middle section 472 than at a first tapered end 474 or a second tapered end 476, as shown in FIGS. 4A, 4B. Alternative embodiments may employ other shape slits, such as, for example, the interior slit 370 in FIG. 3.

FIG. 4B depicts the transducer 400B applied to an example breast site. For ease in explanation, the transducer 400B is depicted on a mannequin. In some embodiments, the transducer may be substantially non-planar. Considering FIG. 4B, and also the associated feature labelling scheme in FIG. 4A, when the transducer 400B is deformed and substantially shaped as a truncated elliptical paraboloid or truncated oblique cone, a substantially circular opening 450 may be formed by the opening 404 (FIG. 4A) at a truncated portion 452 of the truncated elliptical paraboloid or truncated oblique cone. The interior slit 470 may reduce creasing of the transducer 400B when deformed to such a shape and worn on the body. In some embodiments, when the transducer 400B is substantially shaped as a truncated elliptical paraboloid or truncated oblique cone, the substantially circular opening 450 may be formed at the truncated portion 452 of the truncated elliptical paraboloid or truncated oblique cone by removing the gap 410 between two opposing ends, namely between the first end 412 and the second end 414 of the substrate 402 (or layer of anisotropic material 403). In some embodiments, when the transducer 400B is substantially shaped as a truncated elliptical paraboloid or truncated oblique cone, the first end portion 406 abuts, touches, or overlaps the second end portion 408, and/or the second end portion 408 abuts, touches, or overlaps the first end portion 406. As shown in FIG. 4B, the second end 414 of the second end portion 408 overlaps the first end 412 of the first end portion 406. As shown in FIG. 4B, the abutting, touching, or overlapping of the first end portion 406 and the second end portion 408 is situated at a lower portion of the breast of the subject. In such embodiments, transducer 400B may be adapted to be positioned on or around an anatomical feature of a subject, for example a breast (FIG. 4B). The substantially circular opening 450 may coincide with a first location on a subject, for example a nipple, and no electrodes of the transducer array may be located over the nipple.

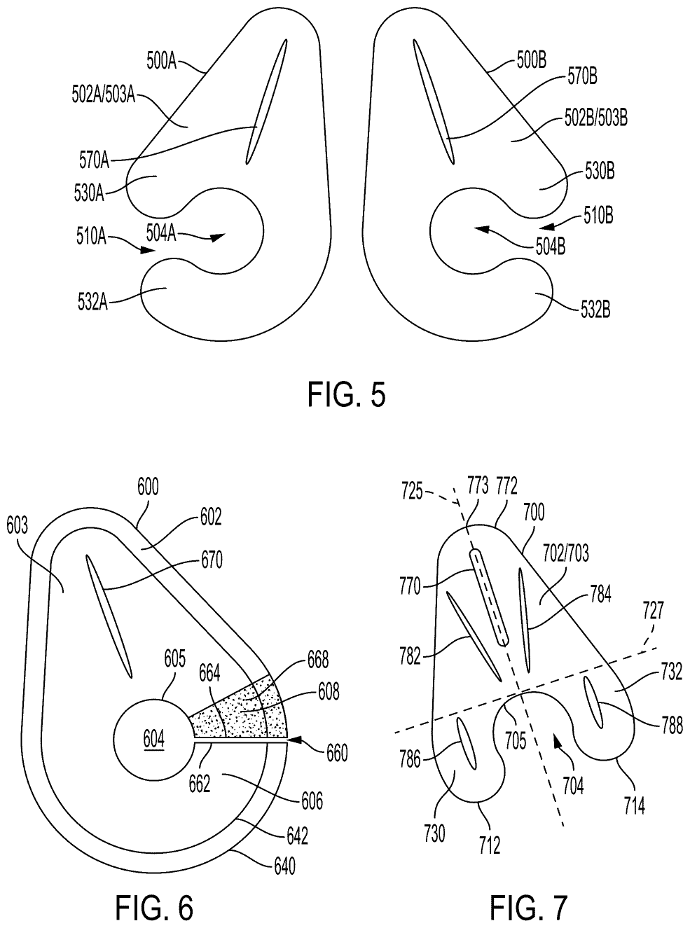

FIG. 5 depicts a top view of two example transducers 500A and 500B. The transducers 500A and 500B are mirror images of each other, but otherwise have the same shape and the same features. The transducers 500A and 500B are similar to the transducer 400A, 400B in FIG. 4A (and follow a similar description and labelling notation) but differ in that each transducer does not having reflectional symmetry, and further as detailed below. The interior slits 570A and 570B are similar to the interior slit 470 in FIG. 4A. In alternative embodiments, the interior slits 570A and 570B may be similar to the interior slit 370 in FIG. 3. The substrates 502A, 502B (or layers of anisotropic material 503A, 503B) have openings 504A, 504B and gaps 510A, 510B, respectively. In some embodiments, the gaps 510A, 510B may be situated on one side of the substrate 502A, 502B (or layer of anisotropic material 503A, 503B). As shown in FIG. 5, when viewed from a direction perpendicular to the face of the substrate 502A and when the transducer 500A is substantially planar, the gap 510A is on the left side of the substrate 502A (or layer of anisotropic material 503A). A first portion 530A is smaller than a second portion 532A. As shown in FIG. 5, when viewed from a direction perpendicular to the face of the substrate 502B (or layer of anisotropic material 503B) and when the transducer 500B is substantially planar, the gap 510B is on the right side of the substrate (or layer of anisotropic material). A first portion 530B is smaller than a second portion 532B. The transducer 500A, 500B may be capable of being deformed from being substantially planar to being substantially non-planar, such as being deformed to be shaped as a truncated elliptical paraboloid or truncated oblique cone in similar manner to that described above for transducer 400A, 400B. Transducer 500A, 500B may similarly be applied to a breast site.

FIG. 6 depicts a top view of an example transducer 600. The transducer 600 is similar to the example transducer 500A, 500B in FIG. 5 and with similar feature labeling, but instead of having a gap 510, the transducer 600 includes a slit 660. The top view in FIG. 6 additionally depicts a layer of anisotropic material 603 which may overlay (and partially obscure) a substrate 602. The substrate 602 may be or may comprise an adhesive bandage to affix the transducer 600 to the subject. When viewed from a direction perpendicular to the face of the transducer 600 and when the transducer 600 is substantially planar, the slit 660 may be located between an exterior edge 640 of the substrate 602 and a concave edge 605 of the opening 604. The slit 660 may separate a first end portion 606 of the transducer 600 from a second end portion 608 of the transducer 600. The slit 660 may be defined by a first edge 662 of the first end portion 606 and a second edge 664 of the second end portion 608. As an example, the slit 660 may be formed by scoring the transducer 600. The first edge 662 and the second edge 664 may both be straight lines between exterior edge 640 of the substrate 602 and the concave edge 605. The slit 660 may be through the layer of anisotropic material 603. The slit 660 may be through the substrate 602. The slit 660 may be through both the substrate 602 and the layer of anisotropic material 603 (as shown in FIG. 6). Similar embodiments may exist for transducers having a substrate but no layer of anisotropic material, or having a layer of anisotropic material but no substrate.

The transducer 600 may include an interior slit 670, which is similar to the interior slits 470 in FIG. 4A and slits 570A and 570B in FIG. 5. Alternatively, the interior slit 670 may be similarly shaped to the interior slit 370 in FIG. 3. The interior slit 670 is different from the slit 660. For example, when viewed from a direction perpendicular to the face of the transducer 600 and when the transducer 600 is substantially planar, the interior slit 670 is surrounded by the layer of anisotropic material 603 (and also the substrate 602). Further, a perimeter of the interior slit 670 does not intersect the edge 642 of the layer of anisotropic material 603, and a perimeter of the interior slit 670 does not intersect the concave edge 605 of the opening 604.

The transducer 600 may further include an overlap portion 668 (the shaded section in FIG. 6) of the second end portion 608. The overlap portion 668 may be designated to overlap the first end portion 606 when the transducer 600 is placed on a subject (e.g., the subject's breast) and deformed from being substantially planar to being substantially non-planar, such as being deformed to be shaped as a substantially truncated elliptical paraboloid or substantially truncated oblique cone.

FIG. 7 depicts a top view of an example transducer 700. The transducer 700 may be structurally similar to transducer 300A, 300B in FIG. 3 and with similar feature labeling, except for having a plurality of interior slits. The transducer 700 may include an interior slit 770, which may be similar to the interior slit 370 in FIG. 3. Alternatively, interior slit 770 may be similarly shaped to the interior slit 470 in FIG. 4A. The transducer 700 may further include interior slits 782 and 784 (shown similarly shaped to the interior slit 470 in FIG. 4A). Alternatively, interior slits 782 and 784 may be similarly shaped to the interior slit 370 in FIG. 3. In some embodiments, the interior slits 782 and 784 may not extend past the concave edge 705 of an opening 704. For example, when viewed from a direction perpendicular to the face of the transducer 700 and when the transducer 700 is substantially planar, the interior slits 782 and 784 may extend from below a top edge 772 (which includes an uppermost point 773) of the substrate 702 (or layer of anisotropic material 703) to above a horizontal line 727 perpendicular to a centerline 725 and intersecting a top of a concave edge 705 of the opening 704. The interior slits 782 and 784 may, or may not, have reflectional symmetry about the centerline 725 running through a longest dimension of the substrate 702. In some embodiments, no electrodes are in the interior slits 782 and 784.

The transducer 700 may further include interior slits 786 and 788 situated within a first portion 730 and a second portion 732, respectively, of the substrate 702 (or layer of anisotropic material 703). A combination of the first portion 730 and the interior slit 786 and a combination of the second portion 732 and the interior slit 788 may be mirror images of each other and may have reflectional symmetry about the centerline 725. In some embodiments, interior slits 786 and 788 do not have reflectional symmetry. As an example, when viewed from a direction perpendicular to the face of the transducer 700 and when the transducer 700 is substantially planar, the interior slits 786 and 788 may extend from at or below the horizontal line 727 to above a first end 712 and a second end 714, respectively. In some embodiments, when viewed from a direction perpendicular to the face of the transducer 700 and when the transducer 700 is substantially planar, one or more interior slits may extend from above the horizontal line 727 (and below the top edge 772) to above a first end 712 and a second end 714, respectively. In some embodiments, no electrodes are in the interior slits 786 and 788.

The interior slits 770, 782, 784, 786, and 788 may extend substantially parallel to the longitudinal direction of the substrate 702 (or layer of anisotropic material 703). In some embodiments, when viewed from a direction perpendicular to the face of the transducer 700, the substrate 702 (or layer of anisotropic material 703) may include at least one interior slit in each quadrant defined by the intersection of the centerline 725 and the horizontal line 727. In some embodiments, when viewed from a direction perpendicular to the face of the transducer 700, the substrate 702 (or layer of anisotropic material 703) does not include an interior slit extending substantially perpendicular to the longitudinal direction of the substrate 702 (or layer of anisotropic material 703).

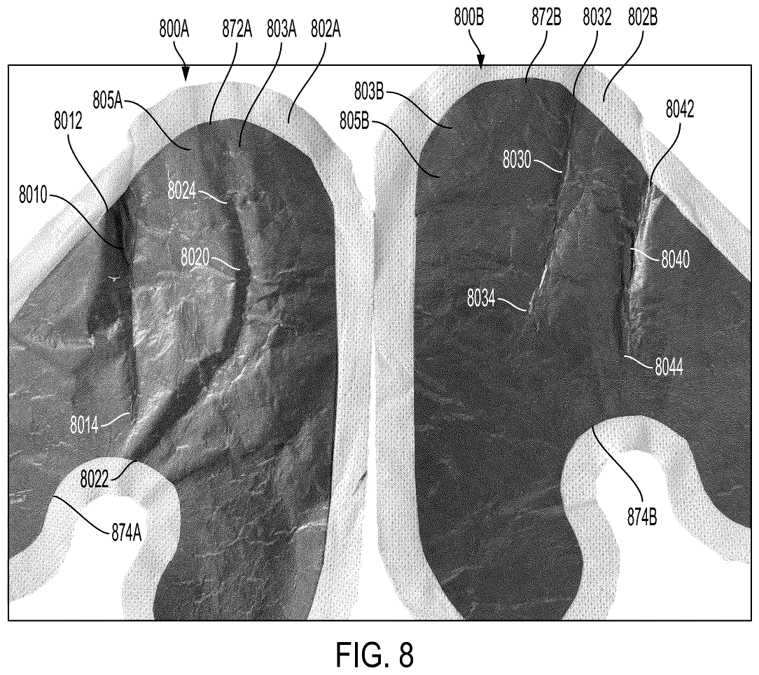

FIG. 8 depicts two transducers 800A and 800B after removal from a subject. The transducers 800A and 800B are examples of the transducers 300A and 300B of FIG. 3 and with similar feature labeling, except without an interior slit 370. The view of the transducers 800A and 800B is of the face of the array facing the subject's body. The transducers 800A and 800B have the same shape and the same features. The transducer 800A, 800B may respectively include a substrate 802A, 802B, an array of at least one electrode (not shown) disposed on the substrate 802A, 802B, and a layer of anisotropic material 803A, 803B. In this example, the substrate may include an adhesive layer for attaching the transducer 800A, 800B to the subject. The at least one electrode (not shown) may be disposed between the substrate 802A, 802B and the layer of anisotropic material 803A, 803B. The layer of anisotropic material 803A, 803B may have a first side 805A, 805B to face the subject and a second side opposite the first side that faces the substrate 802A, 802B.

As can be seen in FIG. 8, both transducer 800A and transducer 800B experience creasing after being applied, worn, and removed from the subject. The transducer 800A includes creases 8010 and 8020. The crease 8010 is generally in a longitudinal direction of the transducer 800A, generally linear in shape, has a first end 8012 at a top edge 872A of the layer of anisotropic material 803A and a second end 8014 within the interior of the layer of anisotropic material 803A, and extends approximately 80% to approximately 90% from the top edge 872A of the layer of anisotropic material 803A to an opposite bottom edge 874A of the layer of anisotropic material 803A. The crease 8020 is generally in a longitudinal direction of the transducer 800A, has a shape similar to a closed parentheses, has a first end 8022 at the bottom edge 874A of the layer of anisotropic material 803A and a second end 8024 within the interior of the layer of anisotropic material 803A, and extends approximately 80% to approximately 90% from the bottom edge 874A of the layer of anisotropic material 803A to the top edge 872A of the layer of anisotropic material 803A.

Transducer 800B includes creases 8030 and 8040. The crease 8030 is generally in a longitudinal direction of the transducer 800B, generally linear in shape, has a first end 8032 at a top edge 872B of the layer of anisotropic material 803B and a second end 8034 within the interior of the layer of anisotropic material 803B, and extends approximately 50% to approximately 60% from the top edge 872B of the layer of anisotropic material 803B to the bottom edge 874B of the layer of anisotropic material 803B. The crease 8040 is generally in a longitudinal direction of the transducer 800B, generally linear in shape, has a first end 8042 at a top edge 872B of the layer of anisotropic material 803B and a second end 8044 within the interior of the layer of anisotropic material 803B, and extends approximately 70% to approximately 80% from the top edge 872B of the layer of anisotropic material 803B to the bottom edge 874B of the layer of anisotropic material 803B.

As recognized by the inventors, creasing may reduce the effectiveness of the electrodes of the transducer 800A, 800B to deliver TTFields, as the surface area of the electrode contacting the skin of the subject may be reduced and/or as the directionality of the TTFields may be affected. As discovered by the inventors, introduction of at least one interior slit to the transducer 800A, 800B may help to reduce creasing of the transducer.

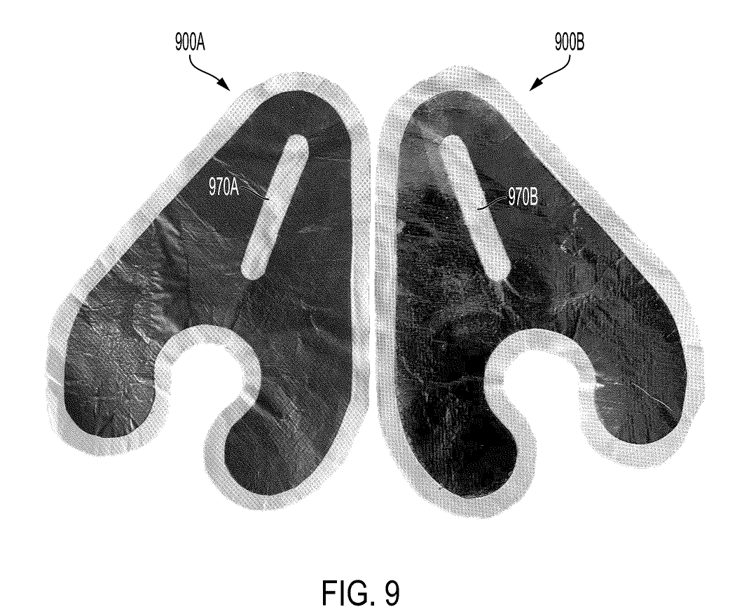

FIG. 9 depicts two transducers 900A and 900B after removal from a subject. The transducers 900A and 900B are examples of the transducers 400A and 400B of FIG. 4 and with similar feature labeling, except the interior slits 970A and 970B are similar to the interior slit 370 of transducers 300A and 300B of FIG. 3. Other example transducers use interior slits similar to slit 470 in FIG. 4A. The view of the transducers 900A and 900B is of the face of the transducers facing the subject's body. The transducers 900A and 900B have a generally similar shape and generally similar features as the transducers 800A and 800B, except the transducers 900A and 900B each have the interior slit 970A and 980B, respectively. The transducer 900A has an interior slit 970A, which is generally in a longitudinal direction of the transducer 900A. The transducer 900B has an interior slit 970B, which is generally in a longitudinal direction of the transducer 900B. As can be seen, and in comparison to the transducers 800A and 800B of FIG. 8, the transducers 900A and 900B do not include creases (e.g., such as creases 8010, 8020, 8030, and 8040 in FIG. 8) due to the inclusion of the interior slits 970A and 970B.

As discussed above, the disclosed transducers may include a layer of anisotropic material located on a skin-facing side of the array of electrode elements (i.e., a side of the array of electrode elements facing the subject's body), as disclosed, for example, in United States Patent Application Publication No. 2023/0037806 A1. The layer of anisotropic material may have anisotropic thermal properties and/or anisotropic electrical properties. If the layer of anisotropic material has anisotropic thermal properties (for example, greater thermal conductivity in the plane of the layer than through the plane of the layer), then the layer spreads heat out more evenly over a larger surface area. If the layer of anisotropic material has anisotropic electrical properties (for example, greater electrical conductivity in the plane of the layer than through the plane of the layer), then the layer spreads the current out more evenly over a larger surface area. In each case, this lowers the temperature of any hot spots and raises the temperature of the cooler regions when a given AC voltage is applied to the array of electrode elements. Accordingly, the current can be increased (thereby increasing the therapeutic effect) without exceeding the safety temperature threshold at any point on the subject's skin.

In some embodiments, the layer of anisotropic material is anisotropic with respect to electrical conductivity properties. In some embodiments, the layer of anisotropic material is anisotropic with respect to thermal conductivity properties. In some preferred embodiments, the layer of anisotropic material is anisotropic with respect to both electrical conductivity properties and thermal conductivity properties.

The anisotropic thermal properties include directional thermal properties. Specifically, the layer of anisotropic material may have a first thermal conductivity in a direction that is perpendicular to its front face (skin-facing surface) that is different from a thermal conductivity of the layer of anisotropic material in directions that are parallel to the front face. For example, the thermal conductivity of the layer of anisotropic material in directions parallel to the front face is more than two times higher than the first thermal conductivity. In some preferred embodiments, the thermal conductivity in the parallel directions is more than ten times higher than the first thermal conductivity. For example, the thermal conductivity of the sheet in directions that are parallel to the front face may be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the layer of anisotropic material may have a first electrical conductivity (or, conversely, resistance) in a direction that is perpendicular to its front face that is different from an electrical conductivity (or resistance) of the layer of anisotropic material in directions that are parallel to the front face. For example, the resistance of the layer of anisotropic material in directions parallel to the front face may be less than the first resistance. In some preferred embodiments, the resistance in the parallel directions is less than half of the first resistance or less than 10% of the first resistance. For example, the resistance of the layer of anisotropic material in directions that are parallel to the front face may be less than: 75%, 50%, 40%, 30%, 20%, 10%, 5%, 1%, 0.5%, or even less than 0.1% of the first resistance.

In some embodiments (e.g., when the layer of anisotropic material is a sheet of pyrolytic graphite), the layer of anisotropic material has both anisotropic electrical properties and anisotropic thermal properties.

### ILLUSTRATIVE EMBODIMENTS

The invention includes other illustrative embodiments (“Embodiments”) as follows.

Embodiment 1. A transducer apparatus for delivering tumor treating fields to a subject's body, the transducer apparatus comprising a substrate; an array of at least one electrode disposed on the substrate, the array configured to be positioned over the subject's body with a face of the array facing the subject's body, the array capable of delivering tumor treating fields to a subject's body; and a layer of anisotropic material positioned on a skin-facing side of the array, wherein, when viewed from a direction perpendicular to the face of the array, the layer of anisotropic material comprises an interior slit extending substantially along a longitudinal direction of the layer of anisotropic material, the interior slit is surrounded by anisotropic material, and no electrodes are in the interior slit.

Embodiment 2: The transducer apparatus of Embodiment 1, wherein, when viewed from a direction perpendicular to the face of the array, the interior slit divides a first portion of the layer of anisotropic material from a second portion of the layer of anisotropic material, and the first portion and the second portion have an approximately same size and an approximately same shape.

Embodiment 3: The transducer apparatus of Embodiment 1, wherein, when viewed from a direction perpendicular to the face of the array, the interior slit is situated along a centerline running through a longest dimension of the layer of anisotropic material.

Embodiment 3A: The transducer apparatus of Embodiment 1, wherein, when viewed from a direction perpendicular to the face of the array, a centroid of the layer of anisotropic material is situated within the interior slit.

Embodiment 4: The transducer apparatus of Embodiment 1, wherein, when viewed from a direction perpendicular to the face of the array, the interior slit comprises approximately 0.5% to approximately 10% of a surface area of the layer of anisotropic material.

Embodiment 4A: The transducer apparatus of Embodiment 1, wherein, when viewed from a direction perpendicular to the face of the array, the interior slit comprises approximately 1% to approximately 5% of a surface area of the layer of anisotropic material.

Embodiment 5: The transducer apparatus of Embodiment 1, wherein, when viewed from a direction perpendicular to the face of the array, the interior slit is approximately 0.5 mm to approximately 10.0 mm wide.

Embodiment 5A: The transducer apparatus of Embodiment 1, wherein, when viewed from a direction perpendicular to the face of the array, the interior slit is approximately 1.0 mm to approximately 5.0 mm wide.

Embodiment 6: The transducer apparatus of Embodiment 1, wherein, when viewed from a direction perpendicular to the face of the array, the interior slit is approximately 1.0 cm to approximately 10.0 cm long.

Embodiment 6A: The transducer apparatus of Embodiment 1, wherein, when viewed from a direction perpendicular to the face of the array, the interior slit is approximately 3.0 cm to approximately 6.0 cm long.

Embodiment 6B: The transducer apparatus of Embodiment 1, wherein the interior slit is a perforation through the layer of anisotropic material.

Embodiment 6C: The transducer apparatus of Embodiment 1, wherein the interior slit has reflectional symmetry.

Embodiment 6D: The transducer apparatus of Embodiment 1, wherein, when viewed from a direction perpendicular to the face of the array, the layer of anisotropic material does not include an interior slit extending substantially perpendicular to the longitudinal direction of the layer of anisotropic material.

Embodiment 7: The transducer apparatus of Embodiment 1, wherein the layer of anisotropic material includes only one interior slit.

Embodiment 8: The transducer apparatus of claim 1, wherein the layer of anisotropic material includes two discontinuous non-parallel interior slits.

Embodiment 8A: The transducer apparatus of Embodiment 1, wherein, when viewed from a direction perpendicular to the face of the array, the layer of anisotropic material comprises a plurality of interior slits extending substantially in parallel and substantially along the longitudinal direction of the layer of anisotropic material.

Embodiment 9: The transducer apparatus of Embodiment 1, wherein the anisotropic material is a sheet of graphite.

Embodiment 9A: The transducer apparatus of Embodiment 1, wherein the anisotropic material is a sheet of pyrolytic graphite, graphitized polymer film, or graphite foil made from compressed high purity exfoliated mineral graphite.

Embodiment 10: The transducer apparatus of Embodiment 1, wherein one or more of the transducer, the layer of anisotropic material, or the substrate has a substantially pear-shaped or rounded triangular-shaped surface having an opening located towards a wider portion of the substantially pear-shaped or rounded triangular-shaped surface.

Embodiment 11: The transducer apparatus of Embodiment 10, wherein the opening defines a substantially C-shaped surface at the wider portion of the substantially pear-shaped or rounded triangular-shaped surface.

Embodiment 11A: The transducer apparatus of Embodiment 1, wherein the transducer apparatus has reflectional symmetry about a centerline running through a longest dimension of the transducer apparatus.

Embodiment 12: The transducer apparatus of Embodiment 1, wherein the transducer apparatus is substantially non-planar, wherein the transducer apparatus is substantially shaped as a truncated elliptical paraboloid or truncated oblique cone, and wherein a substantially circular opening is formed by an opening at a truncated portion of the truncated elliptical paraboloid or truncated oblique cone.

Embodiment 13: The transducer apparatus of Embodiment 1, wherein the substrate comprises an interior slit extending substantially along a longitudinal direction of the layer of the substrate, the interior slit is surrounded by the substrate, and no electrodes are in the interior slit.

Embodiment 14: The transducer apparatus of Embodiment 13, wherein the layer of anisotropic material comprises an interior slit extending substantially along a longitudinal direction of the layer of the substrate, wherein the interior slit in the layer of anisotropic material and the interior slit in the substrate are coincident forming a combined interior slit, and no electrodes are in the combined interior slit.

Embodiment 14A: The transducer apparatus of Embodiment 1, wherein the transducer apparatus is adapted to be positioned on or around an anatomical feature of a subject.

Embodiment 14B: The transducer apparatus of Embodiment 14A, wherein the anatomical feature is a breast.

Embodiment 14C: The transducer apparatus of Embodiment 1, wherein the transducer apparatus is adapted to be positioned on a head of a subject.

Embodiment 14D: The transducer apparatus of Embodiment 1, wherein the transducer apparatus is adapted to be positioned on a torso of a subject.

Embodiment 14E: A transducer apparatus for delivering tumor treating fields to a subject's body, the transducer apparatus comprising a substrate; an array of at least one electrode disposed on the substrate, the array configured to be positioned over the subject's body with a face of the array facing the subject's body; and a layer of anisotropic material positioned on a skin-facing side of the array, wherein, when viewed from a direction perpendicular to the face of the array, the layer of anisotropic material comprises a plurality of interior slits extending substantially in parallel and substantially along a longitudinal direction of the layer of anisotropic material, and the layer of anisotropic material does not include an interior slit extending substantially perpendicular to the longitudinal direction of the layer of anisotropic material.

Embodiment 14F: The transducer apparatus of Embodiment 14E, when viewed from a direction perpendicular to the face of the array, each interior slit is surrounded by anisotropic material, and no electrodes are in the interior slits.

Embodiment 15: A transducer apparatus for delivering tumor treating fields to a subject's body, the transducer apparatus comprising a substrate; and an array of at least one electrode disposed on the substrate, the array configured to be positioned over the subject's body with a face of the array facing the subject's body; wherein, when viewed from a direction perpendicular to the face of the array and when the transducer apparatus is substantially planar, the transducer apparatus has a substantially pear-shaped or rounded triangular-shaped surface having an opening located towards a wider portion of the substantially pear-shaped or rounded triangular-shaped surface, the substrate comprises an interior slit extending substantially along a longitudinal direction of the substrate, and no electrodes are in the interior slit.

Embodiment 15A: The transducer apparatus of Embodiment 15, wherein the interior slit is located closer to a narrower portion than a wider portion of the substantially pear-shaped or rounded triangular-shaped surface.

Embodiment 15B: The transducer apparatus of Embodiment 15, wherein when viewed from a direction perpendicular to the face of the array and when the transducer apparatus is substantially planar, the transducer apparatus comprises a first end portion separated from a second end portion by a gap.

Embodiment 15C: The transducer apparatus of Embodiment 15B, wherein the first end portion has a first edge and a second edge, the first edge defining a portion of an exterior edge of the substantially pear-shaped or rounded triangular-shaped surface, the second edge defining a portion of the opening; and wherein the second end portion has a first edge and a second edge, the first edge defining a portion of the exterior edge of the substantially pear-shaped or rounded triangular-shaped surface, the second edge defining a portion of the opening.

Embodiment 15D: The transducer apparatus of Embodiment 15B, wherein the first end portion and the second end portion each include a portion of the array of at least one electrode.

Embodiment 15E: The transducer apparatus of Embodiment 15B, wherein only one of the first end portion or the second end portion includes a portion of the array of at least one electrode.

Embodiment 15F: The transducer apparatus of Embodiment 15B, wherein the first end portion and the second end portion are part of a single continuous substrate.

Embodiment 15G: The transducer apparatus of Embodiment 15B, wherein the first end portion and the second end portion are located on two separate discontinuous sections of substrate.

Embodiment 16: The transducer apparatus of Embodiment 15, wherein, when viewed from a direction perpendicular to the face of the array and when the transducer apparatus is substantially planar, the substrate has at least one concave edge defining the opening between two opposing sides of the substrate, and the opening defines a substantially C-shaped surface at the wider portion of the substantially pear-shaped or rounded triangular-shaped surface.

Embodiment 16A: The transducer apparatus of Embodiment 15, wherein the substrate has reflectional symmetry.

Embodiment 17: The transducer apparatus of Embodiment 16, wherein a gap defined by the substantially C-shaped surface is situated on one side of the substrate and is defined by a centerline running through a longest dimension of the substrate and through a center of the gap.

Embodiment 18: The transducer apparatus of Embodiment 15, wherein, when the transducer apparatus is substantially non-planar, the transducer apparatus is substantially shaped as a truncated elliptical paraboloid or truncated oblique cone, and a substantially circular opening is formed by an opening at a truncated portion of the truncated elliptical paraboloid or truncated oblique cone.

Embodiment 19: The transducer apparatus of Embodiment 15, wherein the transducer apparatus further comprises a layer of anisotropic material on a skin-facing side of the array.

Embodiment 20: The transducer apparatus of Embodiment 19, wherein the layer of anisotropic material is a sheet of graphite.

Embodiment 20A: The transducer apparatus of Embodiment 19, wherein the layer of anisotropic material is a sheet of pyrolytic graphite, graphitized polymer film, or graphite foil made from compressed high purity exfoliated mineral graphite.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
a substrate;
an array of at least one electrode disposed on the substrate, the array configured to be positioned over the subject's body with a face of the array facing the subject's body, the array capable of delivering tumor treating fields to a subject's body; and
a layer of anisotropic material positioned on a skin-facing side of the array;
wherein, when viewed from a direction perpendicular to the face of the array,
the layer of anisotropic material comprises an interior slit extending substantially along a longitudinal direction of the layer of anisotropic material,
the interior slit is surrounded by anisotropic material, and
no electrodes are in the interior slit.

2. The transducer apparatus of claim 1, wherein, when viewed from a direction perpendicular to the face of the array,
the interior slit divides a first portion of the layer of anisotropic material from a second portion of the layer of anisotropic material, and
the first portion and the second portion have an approximately same size and an approximately same shape.

3. The transducer apparatus of claim 1, wherein, when viewed from a direction perpendicular to the face of the array, the interior slit is situated along a centerline running through a longest dimension of the layer of anisotropic material.

4. The transducer apparatus of claim 1, wherein, when viewed from a direction perpendicular to the face of the array, the interior slit comprises approximately 0.5% to approximately 10% of a surface area of the layer of anisotropic material.

5. The transducer apparatus of claim 1, wherein, when viewed from a direction perpendicular to the face of the array, the interior slit is approximately 0.5 mm to approximately 10.0 mm wide.

6. The transducer apparatus of claim 1, wherein, when viewed from a direction perpendicular to the face of the array, the interior slit is approximately 1.0 cm to approximately 10.0 cm long.

7. The transducer apparatus of claim 1, wherein the layer of anisotropic material includes only one interior slit.

8. The transducer apparatus of claim 1, wherein the layer of anisotropic material includes two discontinuous non-parallel interior slits.

9. The transducer apparatus of claim 1, wherein the anisotropic material is a sheet of graphite.

10. The transducer apparatus of claim 1, wherein one or more of the transducer, the layer of anisotropic material, or the substrate has a substantially pear-shaped or rounded triangular-shaped surface having an opening located towards a wider portion of the substantially pear-shaped or rounded triangular-shaped surface.

11. The transducer apparatus of claim 10, wherein the opening defines a substantially C-shaped surface at the wider portion of the substantially pear-shaped or rounded triangular-shaped surface.

12. The transducer apparatus of claim 1, wherein the transducer apparatus is substantially non-planar,
wherein the transducer apparatus is substantially shaped as a truncated elliptical paraboloid or truncated oblique cone, and
wherein a substantially circular opening is formed by an opening at a truncated portion of the truncated elliptical paraboloid or truncated oblique cone.

13. The transducer apparatus of claim 1, wherein the substrate comprises an interior slit extending substantially along a longitudinal direction of the layer of the substrate, the interior slit is surrounded by the substrate, and no electrodes are in the interior slit.

14. The transducer apparatus of claim 13, wherein the layer of anisotropic material comprises an interior slit extending substantially along a longitudinal direction of the layer of the substrate,
wherein the interior slit in the layer of anisotropic material and the interior slit in the substrate are coincident forming a combined interior slit, and no electrodes are in the combined interior slit.

15. A transducer apparatus for delivering tumor treating fields to a subject's body, the transducer apparatus comprising:
a substrate; and
an array of at least one electrode disposed on the substrate, the array configured to be positioned over the subject's body with a face of the array facing the subject's body;
wherein, when viewed from a direction perpendicular to the face of the array and when the transducer apparatus is substantially planar,
the transducer apparatus has a substantially pear-shaped or rounded triangular-shaped surface having an opening located towards a wider portion of the substantially pear-shaped or rounded triangular-shaped surface,
the substrate comprises an interior slit extending substantially along a longitudinal direction of the substrate, and
no electrodes are in the interior slit.

16. The transducer apparatus of claim 15,
wherein, when viewed from a direction perpendicular to the face of the array and when the transducer apparatus is substantially planar,
the substrate has at least one concave edge defining the opening between two opposing sides of the substrate, and
the opening defines a substantially C-shaped surface at the wider portion of the substantially pear-shaped or rounded triangular-shaped surface.

17. The transducer apparatus of claim 16, wherein a gap defined by the substantially C-shaped surface is situated on one side of the substrate and is defined by a centerline running through a longest dimension of the substrate and through a center of the gap.

18. The transducer apparatus of claim 15, wherein the transducer apparatus is substantially non-planar,
wherein the transducer apparatus is substantially shaped as a truncated elliptical paraboloid or truncated oblique cone, and
wherein a substantially circular opening is formed by an opening at a truncated portion of the truncated elliptical paraboloid or truncated oblique cone.

19. The transducer apparatus of claim 15, wherein the transducer apparatus further comprises a layer of anisotropic material on a skin-facing side of the array.

20. The transducer apparatus of claim 19, wherein the layer of anisotropic material is a sheet of graphite.
